(19) United States
(12) Patent Application Publication
(10) Pub. No.: US 2015/0209191 A1
(43) Pub. Date: Jul. 30, 2015

(54) NOSE BLEED KIT

(71) Applicant: Merle Bruce, Reno, NV (US)

(72) Inventor: Merle Bruce, Reno, NV (US)

(21) Appl. No.: 14/168,915

(22) Filed: Jan. 30, 2014

(51) Int. Cl. A61F 13/20; A61M 31/00; A61F 5/34

(52) U.S. Cl. CPC A61F 13/2005; A61F 5/34; A61M 31/00; A61F 2013/00472

(57) ABSTRACT

A self-contained module is used to stem nose bleeds. A porous dressing, such as a moldable polymer, has a size that allows the dressing, when dry, to be easily inserted into a nostril. The dressing expands when wetted. In one embodiment, an oblong capsule is molded into the dressing and contains a single dose of a vaso-constrictor solution. When a nose bleed is detected, the user breaks the capsule, such as by bending the dressing, and quickly inserts the dressing into a nostril. The solution is thus distributed along the length of the dressing from the inside out. In another embodiment, a squeezable bladder is provided with a conical tip that fits into a conical hole in the bottom of the dressing. The tip wets the dressing from the inside out when the bladder is squeezed, while the dressing is in the nostril. The bladder and tip are then removed.

### FIELD OF THE INVENTION

This invention relates to a device to stop or mitigate nose bleeds and, in particular, to a single use module of a combination of an absorbent nasal dressing and a dispenser containing a vaso-constrictor solution.

### BACKGROUND

Some people suffer from nose bleeds and may find it important to carry with them an absorbent material, such as a cotton ball or a shaped sponge, along with a multiple-use bottle of a vaso-constrictor solution, such as neosynephrine or oxymetazoline. The absorbent material is generally referred to as a dressing. Upon sensing a nose bleed, the user inserts the dressing in a nostril, bends her head backwards, and dispenses a few drops of the vaso-constrictor solution from the bottle into the exposed end of the dressing. If enough solution is applied, the solution wicks up throughout the dressing, causing it to expand and contact the walls of the nostril to stop the bleeding. Such a procedure is embarrassing, cumbersome, time-consuming, prone to error by dispensing too much or too little solution, primarily wets the bottom of the dressing, and requires the person to carry around more vaso-constrictor solution than what would be needed for the day.

As a medical doctor, I have patients that suffer from nose bleeds. I have frequently distributed to those patients a package of a commercially available Merocel™ sponge nasal dressing, by Medtronic, Inc., along with a bottle of a common vaso-constrictor solution to apply in the manner described above. The patient would then carry around some of the dressings and an amount of the solution in the event of an unexpected nose bleed.

FIGS. 1A and 1B illustrate one type of the Merocel™ sponge nasal dressing 10, where FIG. 1A is a front view and FIG. 1B is a top down view. The dressing 10 is highly absorbent and expands rapidly when wetted as the liquid flows through the dressing 10 by capillary action. The dressing 10 comprises polyvinyl acetate (PVA), which is a moldable porous polymer. Detailed information regarding the dressing 10 is found at http://www.merocel.com/products/index.htm, incorporated herein by reference. The dressing 10 is narrow enough to easily fit into a nostril, and expands to the walls of the nostril when the user dispenses a sufficient amount of the vaso-constrictor solution.

A string 12 is attached to the dressing 10 to eventually remove the dressing 10 from the nostril.

What is needed is a better solution to stop nose bleeds.

### SUMMARY

In one embodiment, the porous Mercel™ sponge material (PVA), or other suitable absorbent material, is molded around an oblong, breakable capsule containing an optimal amount of vaso-constrictor solution for a single use. The combination of the dressing and the capsule is referred to herein as a module. In another embodiment, the capsule is inserted into a hole coaxial with the dressing. Upon the user sensing a nose bleed, the user removes the module from a small kit containing a portable supply of modules, bends or squeezes the dressing to break the capsule and release the solution into the dressing from the inside out, and quickly inserts the dressing into a nostril. The capsule may be provided with a weak lengthwise seam that easily breaks and releases the solution along the entire length of the dressing. In another embodiment, the capsule is provided with many distributed weak areas which open when the capsule is squeezed. The solution spreads out uniformly from the center axis of the dressing. The dressing will then expand in the nostril. Since the dressing itself includes the solution, and an optimal amount of solution is released, the process is quick, discreet, and effective.

A loose plastic bag may be supplied around the dressing to allow the user to squeeze or bend the dressing with the user's fingers to break the capsule without directly touching the dressing. The plastic bag is progressively removed as the user inserts the dressing into a nostril. This reduces the risk of contamination.

In another embodiment, the dressing has a conical hole in the bottom that extends at least half way into the dressing. A small, disposable, squeezable bladder containing a single use of the solution is provided that has a relatively long conical tip that precisely fits in the hole in the dressing. The tip includes one or more holes along its length or is porous. The dressing is supplied in a kit with the bladder tip already inserted into the hole, as a module. Upon the user sensing a nose bleed, the user removes the module from the kit and uses the bladder to push the dressing into a nostril. The user then squeezes the bladder to forcibly inject the full amount of solution into the dressing, at which time the dressing will expand to retain the dressing in the nostril. The bladder, along with its tip, is then removed and disposed of. In this way, the user does not have to touch the dressing, but may only touch the bladder, creating a very sanitary application.

In one embodiment, the user may refill the bladders, such as while at home, by squeezing and releasing an empty bladder while the tip is immersed in the solution. The solution then fills the bladder by vacuum pressure when the bladder expands.

In both embodiments, the solution is evenly dispersed along the length of the dressing inside out, only the proper amount of solution is applied, and the application is easy to use. The user does not need to tip her head back when applying the solution since gravity is not used to distribute the solution.

Other embodiments are described.

### BRIEF DESCRIPTION OF THE DRAWINGS

FIG. 1A is a front view of a prior art dressing intended to be inserted into a nostril to stop nose bleeds.

FIG. 1B is a top down view of the dressing of FIG. 1A.

FIG. 2 is a bisected view of a dressing that contains a breakable capsule containing a vaso-constrictor solution, in accordance with one embodiment of the invention.

FIG. 3 is a flowchart describing the steps for use of the module of FIG. 2.

FIG. 4 illustrates the module of FIG. 2 or FIG. 6 being inserted into the user's nostril at the onset of a nose bleed.

FIG. 5 illustrates the module of FIG. 2 supplied in a portable day kit.

FIG. 6 is a bisected view of a dressing having a conical hole in its base, and also illustrates a bladder with a conical tip that fits into the hole, in accordance with another embodiment of the invention.

FIG. 7 is a flowchart describing the steps for use of the module of FIG. 6.

FIG. 8 illustrates the module of FIG. 6 supplied in a portable day kit.

FIG. 9 is a bisected view of a dressing with a coaxial hole for receiving a thin breakable capsule containing the vaso-constrictor solution, in accordance with another embodiment of the invention.

FIG. 10 illustrates how the dressing may be supplied in a plastic bag to allow the user to manipulate the dressing, such as breaking the capsule and inserting the dressing into a nostril, without direct contact of the user's fingers with the dressing.

Elements that are similar or identical in the various figures are labeled with the same numeral.

### DETAILED DESCRIPTION

FIG. 2 is a bisected view of a moldable dressing 16 that contains an oblong, breakable capsule 18 containing a vaso-constrictor solution, in accordance with one embodiment of the invention. Other shapes of the capsule may instead be used. The combination of the dressing 16 and capsule 18 forms a single-use module. The solution may be neosynephrine, oxymetazoline, or any other suitable solution for constricting blood vessels and stopping the flow of blood.

The capsule 18 is a thin, breakable plastic, or other suitable material, which keeps the dressing 16 dry until broken. The capsule 18 may be molded to have a weak area along its surface, such as along its entire length, that breaks open upon a slight bending or compression of the dressing 16 by the user's thumb and fingers. In another embodiment, the capsule 18 is molded to have many weak areas that break or leak the solution when the capsule is squeezed or bent. The capsule 18 is designed so that the solution wets the dressing 16 along at least its center section to provide the most uniform distribution of the solution.

In one embodiment, the capsule 18 is a molded plastic formed by two lengthwise halves. The solution fills the halves, and the edges of the halves are adhered together, such as by an adhesive or heat. The adhesion of the two halves is formed weak so the capsule 18 can easily be broken, and the break extends the entire length of the capsule 18 for maximum wetting of the dressing 16 along its length. The capsule 18 is preferably oblong with a length greater than half the length of the dressing 16.

In one embodiment, the dressing 16 is formed of the same or similar porous polymer used to form the Merocel™ sponge nasal dressing, by Medtronic, Inc. Any other suitable dressing materials may instead be used.

To form the dressing 16, a clamshell type mold is provided that molds two connected halves of the dressing 16 with a central indentation for the capsule 18. After molding, such as using injection molding or compression molding, the capsule 18 is placed in the indention, the two halves of the dressing 16 are brought together to enclose the capsule 18, and the abutting surfaces of the dressing halves are affixed together with an adhesive or by heat. One skilled in the art of molding can easily form the dressing 16 containing the capsule 18. Other shapes of capsules and their location within the dressing are also contemplated. Capsules containing any liquids are commonly formed.

The external features of the dressing 16 may be identical to the features of the prior art dressing 10 sold by Medtronic, Inc. The size of the dressing 16, when dry, is narrow enough to easily fit into a nostril without significant pushing force.

FIG. 3 is a flowchart describing the use of the module of FIG. 2. In step 20, the breakable capsule 18 is molded into the dressing 16 and carried by the user as a single-use module. In step 22, upon the user sensing the onset of a nose bleed, the user then bends the dressing 16 or compresses it to break the capsule 18 and begin wetting the dressing 16. In step 24, the user quickly inserts the dressing 16 in the user's nostril 25, as shown in FIG. 4, prior to the dressing 16 significantly expanding. The dressing 16 then evenly wets and expands to tightly fit in the nostril and remains in the nostril. The string 12 may be used to pull out the dressing 16 after the nose bleed has stopped.

In another embodiment, the capsule 18 does not break but is porous or has small holes in its walls. The capsule 18 does not allow the solution to leak out into the dressing 16, due to the small size of the openings or the permeable nature of the capsule, unless the capsule 18 is squeezed or bent. This may allow the dressing 16 to be inserted into the nostril, followed by the user squeezing her nose to compress the capsule 18 and release the solution into the dressing 16. Instead, the user may squeeze or bend the dressing prior to insertion into the nostril, but must then quickly insert it before the dressing significantly expands.

FIG. 5 illustrates an easily opened kit 30, sold as a unit, containing a pocket-size supply of the modules of FIG. 2, such as four modules. The packaging protects the dressings 16 and keeps them sanitary.

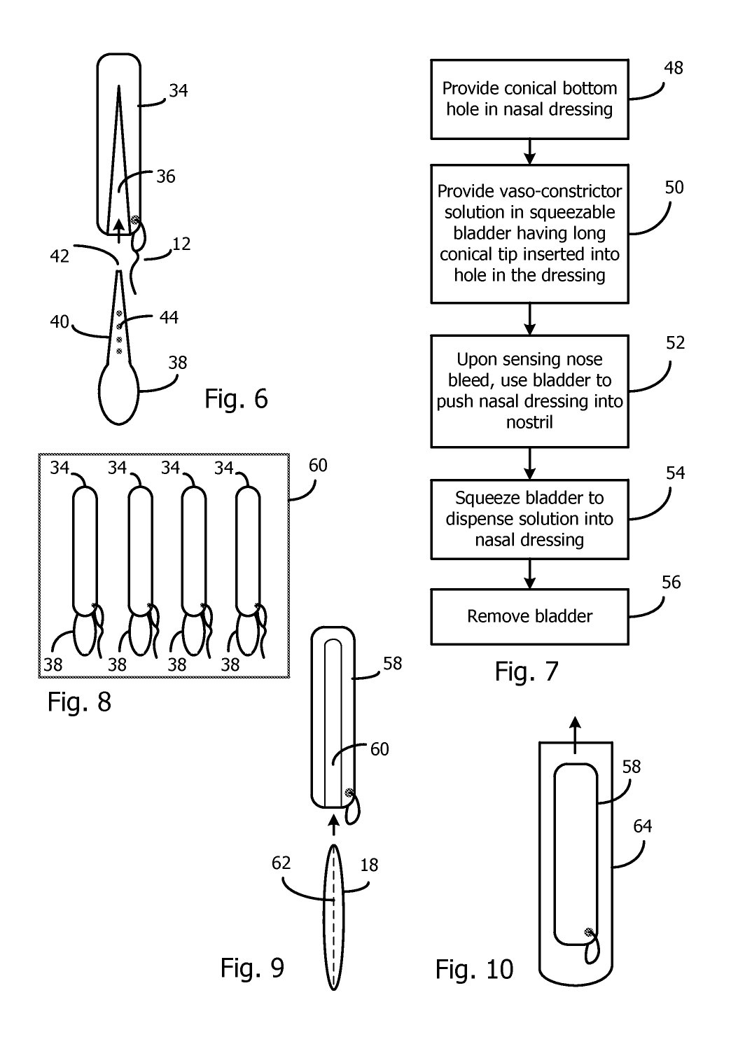

FIG. 6 is a bisected view of a dressing 34 in accordance with another embodiment of the invention. In FIG. 6, the dressing 34 material may be the same moldable porous polymer described above or any other type of suitable porous material.

The dressing 34 is molded to have a generally conical hole 36 formed in its bottom, otherwise the dressing 34 may be substantially identical to the prior art dressing 10. The string 12 may be attached to a side area of the dressing 34 so as not to enter the hole 36 area. The hole 36 should extend at least half the length into the dressing 34, and preferably at least ¾ of the length for even wetting along its length.

A small, squeezable, plastic bladder 38 is provided that contains a single dose of the vaso-constrictor solution. Forming squeezable bladders, of other shapes, containing a liquid for other uses are known. The bladder 38 is provided with a conical tip 40 that is about the length and size of the hole 36, so the tip 40 fits easily into the hole 36 with little or no friction. The tip 40 has an opening 42 at its end and other openings 44 around its length for distributing the solution uniformly throughout the dressing 34 when the bladder 38 is squeezed. Alternately, the tip 40 is porous so that the solution can exit from all areas of the tip 40.

FIG. 7 is a flowchart describing the use of the module of FIG. 6. In step 48, the dressing 34 is provided with the conical hole 36, such as by molding. In step 50, the bladder 38, containing the solution, is initially provided in a kit, where the tip 40 of the bladder 38 is already inserted into the hole 36. In step 52, upon sensing the onset of a nose bleed, the user removes the bladder/dressing 34 from the kit, and uses the bladder 38 as a handle to insert the dressing 34 into a nostril 25 (FIG. 4). By the user having only minimal finger contact with the dressing 34, the dressing 34 remains sanitary. In step 54, the user squeezes the bladder 38 so that the solution exits through the openings 42/44 along the length of the tip 40 and uniformly wets the dressing 34 from the inside out. The dressing 34 then expands to firmly remain in the nostril 25. In step 56, the user then easily removes the bladder 38 and tip 40 from the hole 36 due to the conical shape and low friction between the surfaces.

In another embodiment, the tip 40 is molded into the dressing 34, and the bladder 38 can be easily snapped off the tip 40 after the solution is dispensed in the dressing 34 while the dressing 34 is in the nostril.

FIG. 8 illustrates an easily opened kit 60, sold as a unit, containing a pocket-size supply of the modules of FIG. 6, such as four modules, having the bladder tips 40 (FIG. 6) already inserted into the holes 36. The packaging protects the dressings 34 and keeps them sanitary.

FIG. 9 is a bisected view of a dressing 58 with a coaxial hole 60 for receiving a thin breakable capsule 18 containing the vaso-constrictor solution, in accordance with another embodiment of the invention. The capsule 18 is inserted in a sanitary manner, such as by a machine or a gloved hand, during the manufacture of the module. The capsule 18 may contain a weak seam 62 that easily breaks when the capsule 18 is compressed to distribute the solution along the length of the dressing 58. The capsule 18 may be broken before or after the dressing 58 is inserted into a nostril.

FIG. 10 illustrates how the dressing 58 may be supplied in a plastic bag 64 to allow the user to manipulate the dressing 58, such as breaking the capsule 18 (FIG. 9) and inserting the dressing 58 into a nostril, without direct contact of the user's fingers with the dressing. The bag 64 is progressively removed as the dressing 58 is inserted into the nostril and disposed of.

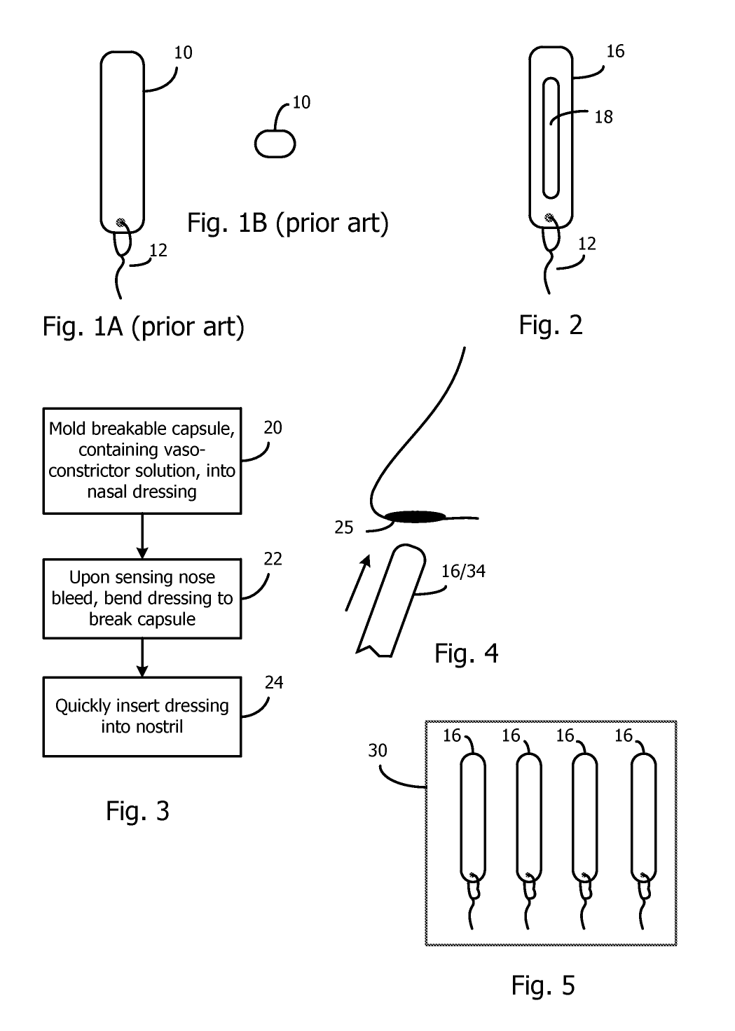

Various other embodiments of the invention are envisioned where the dressing is pre-mated with a dispenser containing a single-use dose of the solution, and where the application of the solution wets the dressing along a substantial portion of its length, rather than just at the bottom as is done in the prior art technique described with respect to FIG. 1. Thus, no extra supply of the solution needs to be carried by the user, and the precise amount is always dispensed. The application of the dressing is very fast and discreet, avoiding embarrassing situations.

Various sizes of the dressings may be provided for children and adults.

Such a system may also be applicable to non-nose bleed uses, such as any situation that requires an initially small piece of porous material to be expanded upon contact with a liquid to form a temporary plug. Such a system may be used for tightly filling holes, or other non-medical or medical uses. In a non-medical use, the liquid may be water. If the plug is to be permanent, the liquid may be a material that does not significantly evaporate over time. The liquid may even be an adhesive material that expands the porous material and then becomes cured to fix the porous material in its expanded size. Various sizes of the materials may be provided, depending on the application.

In another embodiment, the module may be for uses other than for tightly fitting into a hole.

While particular embodiments of the present invention have been shown and described, it will be obvious to those skilled in the art that changes and modifications may be made without departing from this invention in its broader aspects and, therefore, the appended claims are to encompass within their scope all such changes and modifications as fall within the true spirit and scope of this invention.

1. A nose-bleed mitigation system comprising:
a porous dressing having a size that allows the dressing to be inserted into a nostril, the dressing being formed of a material that expands when wetted, the dressing having a cavity;
a dispenser configured to be at least partially positioned in the cavity in the dressing; and
a vaso-constrictor solution contained in the dispenser, wherein the dispenser is configured to dispense the solution from one or more openings of the dispenser into the cavity in the dressing when manually manipulated by a user, such that the dressing is wetted from the inside out, and wherein the dressing expands while in the nostril after wetting by the solution.

2. The system of claim 1 wherein the dispenser comprises a capsule containing the solution, wherein the capsule is contained in the cavity in the dressing.

3. The system of claim 2 wherein the capsule is breakable upon bending the dressing to release the solution into the dressing.

4. The system of claim 2 wherein the capsule releases the solution into the dressing upon squeezing the capsule.

5. The system of claim 2 where the capsule has a length over one half a length of the dressing.

6. The system of claim 2 wherein the dressing is formed of a moldable polymer material, and the dressing is molded to contain the cavity.

7. The system of claim 1 wherein the dressing, dispenser, and solution comprise a first module in a kit containing a plurality of identical other modules.

8. The system of claim 1 wherein the cavity in the dressing comprises a hole in a bottom surface of the dressing, wherein the hole extends partially into the dressing, wherein the dispenser comprises a squeezable bladder containing the solution, wherein the bladder has a tip that fits into the hole, and wherein the tip has one or more openings where the solution exits when the bladder is squeezed.

9. The system of claim 8 wherein the hole in the dressing is generally conical, and the tip is also generally conical.

10. The system of claim 8 wherein the hole extends at least half a length of the dressing.

11. The system of claim 8 wherein the tip has a plurality of openings along a length of the tip.

12. The system of claim 8 wherein the tip is removable from the hole after the dressing has expanded in the nostril.

13. A method for mitigating nose bleeds comprising:
providing a dry porous dressing having a size that allows the dressing to be inserted into a nostril, the dressing being formed of a material that expands when wetted, the dressing having a cavity;
providing a dispenser at least partially positioned in the cavity in the dressing;
providing a vaso-constrictor solution contained in the dispenser; and
manipulating the dispenser to dispense the solution from one or more openings of the dispenser into cavity in the dressing, such that the dressing is wetted from the inside out, and wherein the dressing expands while in the nostril after wetting by the solution.

14. The method of claim 13 wherein the dispenser comprises a capsule containing the solution, wherein the capsule is contained in the cavity in the dressing, and wherein the step of manipulating the dispenser comprises compressing the capsule to force the solution to be released from the capsule inside the dressing.

15. The method of claim 14 wherein manipulating the dispenser comprises breaking the capsule.

16. The method of claim 13 wherein the cavity in the dressing comprises a hole in a bottom surface of the dressing, wherein the hole extends partially into the dressing, wherein the dispenser comprises a squeezable bladder containing the solution, wherein the bladder has a tip that fits into the hole, wherein the tip has the one or more openings, and wherein the step of manipulating the dispenser comprises squeezing the bladder while the tip is inserted in the hole and while the dressing is inserted into the nostril so that the dressing is wetted along a length from the inside out.

17. The method of claim 16 further comprising removing the tip from the hole while the dressing is in the nostril.

18. A plugging system comprising:
a porous plug, the plus being formed of a material that expands when wetted, the plug having a cavity;
a dispenser configured to be at least partially positioned in the cavity in the plug; and
a liquid contained in the dispenser, wherein the dispenser is configured to dispense the liquid from one or more openings of the dispenser into the cavity in the plug when manually manipulated by a user, such that the plug is wetted from the inside out, and wherein the plug expands after wetting by the liquid.

19. The system of claim 18 wherein the dispenser comprises a capsule contained in the cavity in the plug.

20. The system of claim 18 wherein the cavity comprises a hole in a bottom surface of the plug, wherein the dispenser comprises a bladder having a tip that is inserted into the hole in the plug.

21. The system of claim 1 wherein only a single dose of the vaso-constrictor solution is contained in the dispenser.

22. The method of claim 13 wherein only a single dose of the vaso-constrictor solution is contained in the dispenser.

23. The system of claim 18 wherein only a single dose of the vaso-constrictor solution is contained in the dispenser.
